(19) United States
(12) Patent Application Publication
(10) Pub. No.: US 2013/0122028 A1
(43) Pub. Date: May 16, 2013

(54) NOVEL VACCINE THAT TARGETS TUMOR VESSELS AS AN EFFICIENT TOOL IN TUMOR THERAPY

(71) Applicants: Anna-Kariu Olsson, Uppsala (SE); Lars Hellman, Uppsala (SE)

(72) Inventors: Anna-Kariu Olsson, Uppsala (SE); Lars Hellman, Uppsala (SE)

(21) Appl. No.: 13/516,385

(22) PCT Filed: Dec. 15, 2010
(86) PCT No.: PCT/SE2010/000300

§ 371 Date: Dec. 27, 2012

(30) Foreign Application Priority Data
Dec. 15, 2009 (SE) 0901565-2

(51) Int. Cl. C07K 14/78; C07K 14/705; C07K 14/47

(52) U.S. Cl. CPC C07K 14/78; C07K 14/4721; C07K 14/705; C07K 14/7056
USPC 424/185.1

(57) ABSTRACT

The invention relates to a composition for the treatment of various cancers. The composition is a vaccine containing sequences of EDB, EDA, annexin A1, endosialin, C domain of tenascin C or magic roundabout (MR) or fragments thereof as single or in a combination coupled to one or several heterologous foreign carrier molecules. The vaccine will produce antibodies that are directed against self proteins which are preferentially expressed in and around tumor vessels. The vaccine is preferably administrated together with an adjuvant.

A novel vaccine that targets tumor vessels as an efficient tool in tumor therapy.

The present invention relates to a novel method designed to stimulate the immune system to produce antibodies that almost exclusively targets tumor vessels. These antibodies, that are directed against self proteins that are preferentially expressed in and around tumor vessels results in an immune attack on the tumor vessels and thereby induce a marked reduction in tumor growth. Although the invention generally relates to a vaccine for use in a mammal, preferred embodiments relates to a vaccine for the use in human, dog cat or horse, the invention will be described generally and with reference to such vaccines for human, feline, canine and equine use.

### BACKGROUND OF THE INVENTION

Angiogenesis—formation of new capillary blood vessels—is essential during development and physiological conditions that require angiogenesis, such as wound healing and the menstrual cycle. However, in healthy males, angiogenesis is rare and the turnover time of the endothelial cell pool is several hundred days. Prolonged and excessive angiogenesis has, however, been implicated in a number of pathological processes, i.e rheumatoid arthritis, retinopathy and tumor growth. The normal vasculature is tightly regulated by a balance between naturally occurring pro- and antiangiogenic factors. One very important such factor is vascular endothelial growth factor (VEGF), which is required for development of a vascular system during embryogenesis and is also a central regulator of adult neovascularization [1].

To date, a number of endogenous factors negatively regulating angiogenesis have been described [2]. The inhibitors described so far mainly fall into three groups; plasma proteins, basement membrane proteins and serine protease inhibitors (serpins). Examples of endogenous inhibitors of angiogenesis are thrombospondin (TSP), a basement membrane protein, endostatin, a fragment of collagen XVIII, angiostatin, a fragment of plasminogen and tumstatin, a fragment of collagen IV. Their mechanisms of action involve induction of endothelial cell apoptosis and/or interference with integrin function, either by utilizing integrins as receptors or by specifically binding to different matrix components. Genetic evidence further supports a role for these molecules as negative regulators of angiogenesis. Over-expression of TSP-1 in the skin of mice results in delayed and reduced skin carcinogenesis, while mice that lack TSP-2 have increased skin vascularization and show enhanced carcinogenesis [3]. Moreover, mice lacking expression of either tumstatin or endostatin show accelerated tumor growth and vascularization [4].

Antiangiogenesis as a clinical strategy to treat diseases characterized by excessive angiogenesis is attractive in many ways, especially when combined with chemotherapy. Starvation of a tumor through reduced vascularization does not target the tumor compartment and hence does not depend upon a specific trait of the tumor cells, which are known to be genetically unstable. To date, five antiangiogenic drugs, which all target either VEGF or VEGFR2, have been approved by the U.S. Food and Drug Administration (FDA) [1]. Clinically, the most successful so far is the VEGF-neutralizing antibody Bevacizumab (Avastin), which was approved early 2004. When given in combination with chemotherapy, Avastin prolongs the survival of patients with metastatic colon cancer, lung and breast cancer. The second antiangiogenic drug approved by the FDA is the anti-VEGF aptamer Pegaptanib/Macugen, which is used in treatment of age-related macular degeneration (AMD). In addition, the receptor tyrosine kinase inhibitors Sunitinib (Sutent) and Sorafenib (Nexavar), targeting VEGFR2 (as well as PDGF-receptor β, Flt3 and c-Kit) was approved in January 2006 and December 2005, respectively, for the treatment of renal and gastrointestinal cancer. The fifth drug targeting VEGF-induced signalling is the VEGF-neutralizing antibody fragment Ranibizumab (Lucentis), approved in June 2006 by the FDA for the treatment of age related macula degeneration, the leading cause of blindness in the Western world.

It was previously assumed that antiangiogenic treatment, since it targets the non-transformed cells in a tumour, would not suffer from problems related to drug resistance, commonly associated with chemotherapy. This hypothesis has, however, proven not entirely correct. Accumulating data show that other angiogenic pathways can replace VEGF as disease progresses [5]. Therefore it is important to develop a variety of antiangiogenic drugs for clinical use, targeting different functions of the angiogenic vessels, comparable to the battery of cytotoxic drugs used by oncologists today.

Several of these above described methods are very costly, the drugs need to be injected intravenously or intramuscularly several times a week, every day or sometimes several times a day to give maximal effect, and does frequently also show effects only in very limited numbers of cancer forms. New drugs that are more easily administrated, more cost-effective and with a more broad spectrum of tumour forms are therefore needed.

Vaccination is an attractive approach for many types of diseases. Broad vaccination programs have served to virtually eradicate disabling and life-threatening conditions such as polio, diphtheria and smallpox. The possibility to use vaccination as a treatment strategy also for cancer has for some time been the focus of intense research. So far there is however no vaccination in clinical use for this disease. There are several reasons why development of vaccines for cancer treatment is more difficult. First, the antigen to be targeted is very often a self-antigen, maybe expressed also under physiological conditions, but at lower levels or during embryogenesis. Therefore, there is a need to break the self-tolerance of the immune system towards the antigen, in contrast to vaccination against a foreign virus or bacterial antigen. Second, tumor cells have developed mechanisms to evade recognition by the immune system, further complicating vaccination against tumor cell antigens. Third, when vaccinating against self-antigens it is important to use disease-specific targets.

We here present a new strategy to enhance the efficiency of tumour vaccines by coupling the self-antigen, the tumour vascular target, to a non-self antigen that is of size large enough to contain a substantial number of T cell epitopes. This ensure the recognition by T cells in almost any MHC background and a vaccine that can be used in an outbread population like humans. The preferential targeting of vessels also reduces the potential of the tumour cells to become resistant to the treatment.

Moreover, antiangiogenic vaccination may provide a cost-effective alternative to repeated and long-term administration of a drug. For individuals at high risk to develop hereditary forms of cancer, antiangiogenic vaccination could also serve as a preventive treatment.

The antigens we have selected for the development of a cancer vaccine are six different antigens that are preferentially expressed in tumor vasculature during adult life, the extra-domain B of fibronectin (EDB) [6], the extra-domain A of fibronectin (EDA) [6], the extra-domain C of tenascin-C [7], annexin Al [8], endosialin [9, 10] and magic roundabout (MR) [11]. EDB is a 91 amino acid domain inserted into fibronectin by alternative splicing. EDB is expressed during embryogenesis, but not in the adult under normal conditions. However, EDB is highly expressed in a number of solid tumors [6]. Targeting of EDB by administration of antibodies coupled to radioactive or cytotoxic agents, have resulted in very promising results in mouse models of cancer and also in a phase II clinical trial (for updated information see http://www.philogen.com/). EDA is another extra domain that also is inserted into fibronectin by alternative splicing (REF). This domain has been seen to be expressed in certain tumor tissues but generally not in other tissues of adults [6]. The C-domain of tenascin C is over-expressed in various tumor types, for instance in high-grade astrocytomas, but undetectable in most normal adult tissues [7]. Likewise MR is expressed at sites of active angiogenesis, like tumors, but not in normal tissue except during embryogenesis [7]. Similarly both endosialin and annexin Al are preferentially expressed in tumor tissue.

### THE PRIOR ART

Vaccination strategies to treat cancer have been a very active area of research both from the pharmaceutical industry and from academic researchers. However the success have been very limited. One very important factor for this lack of success has been that most clinical and preclinical tests have been performed with unmodified protein. This has also resulted in that almost all groups within the tumor biology field has come to the conclusion that it is not possible to obtain active tumor vaccines. Several groups and companies have therefore turned to use monoclonal antibodies. Even more difficult has the situation been with EDB where the sequence is so extremely well conserved that it is almost impossible to make monoclonal antibodies in mice or rats against the human protein, that they have used phage display technology to obtain EDB binding structures for therapy (see http://www.philogen.com/). There is however at least one company that has made studies of cancer vaccines with modified proteins that is Pharmexa in Denmark. They have used so called immunodominat epitopes from various non-self proteins. These short immundominant epitopes have been inserted in the target molecule (www.pharmexa.com). The idea behind the insertion of immunodominant epitopes is, however, that it is an epitope that binds very strongly to a particular MHC class I, or class II molecule. This makes these vaccines dependent on the highly varying parameter, the peptide specificity of the MHC molecules. By using fusion proteins with larger number of peptides we overcome the problem with single immunodominant epitopes and obtain a vaccine with more general use. All previously tested vaccines have also targeted the tumor and not vessel associated antigens. Tumor antigens have been shown to be easily down-regulated and the tumor may thereby evade these treatment methods relatively easy. This is due to the high plasticity of the tumor cell. All tumors do, however, need vessels to survive and grow and this strategy of targeting vessels with a new more potent therapeutic vaccine consisting of a single or a mix of up to six different tumor specific vascular targets is therefore a novel and more powerful way to target even aggressive fast growing tumors.

### OBJECT OF THE INVENTION

The object of the invention is to provide a convenient, efficient and cost effective method to treat various types of cancers by targeting tumor vasculature, an essential part of a growing tumor. Treatment with vaccines consisting of fusion proteins between the extrecellur domain B (EDB) of fibronectin, EDA, annexin Al, endosialin, the extra domain C of tenascin C or magic roundabout (MR) and a foreign carrier molecule of any non self origin, either one at a time or a combination of two to six of these fusion proteins for the treatment of various forms of cancers.

### SUMMARY OF THE INVENTION

The above described objective is achieved according to the invention by a vaccine or a combination of vaccines, which are characterized by containing one or a combination of two to six proteins having the entire amino acid sequence of EDB, EDA, annexin A1, endosialin, the extra domain C of tenascin C or magic roundabout (MR) from the species to be vaccinated or a segment larger than 5 amino acids of said amino acid sequence in its original or multimerized form coupled to one or more heterologous carrier proteins and by optionally containing an adjuvant.

### BRIEF DESCRIPTION OF THE FIGURES

FIG. 1 A, B, C, D, E and F shows the amino acid sequences of human EDB, EDA, annexin A1, endosialin, the extra domain C of tenascin C or magic roundabout (MR), respectively.

FIG. 2 shows a schematic representation of the thioredoxin—EDB and GST—EDB construct used for immunization, as ELISA coating antigen and for studies of proof of concept in a mouse tumor model, and the purified protein from the same constructs.

FIG. 3 shows the effect on tumor weight after vaccination. The antibody titers in a panel of vaccinated and control mice are presented.

FIG. 4 shows the marked effect on tumor size by the vaccination strategy in a mouse tumor model.

### DESCRIPTION OF THE INVENTION

Anti- EDB, anti-EDA, anti-annexin A1, anti-endosialin, anti-the extra domain C of tenascin C or anti-magic roundabout (MR) antibodies are produced in the host by active immunization, so called vaccination. By injection of modified EDB, EDA, annexin Al, endosialin, the extra domain C of tenascin C or magic roundabout (MR) into the host the immune system of the host produces a polyclonal antibody response against its own EDB, EDA, annexin A1, endosialin, the extra domain C of tenascin C or magic roundabout (MR) and thereby targeting the tumor specific vessels for attack by the immune system. It is of major importance to modify the antigen so that the immune system of the host recognize the modified self-protein as a non-self protein. This can be achieved by covalent coupling of non-self amino acid regions to EDB, EDA, annexin A1, endosialin, the extra domain C of tenascin C or magic roundabout (MR) or selected regions of any of these three molecules from the species to be treated. The peptides within the non-self region then attract and activate non-tolerized T cells, which give help for the potentially auto-reactive B cells.

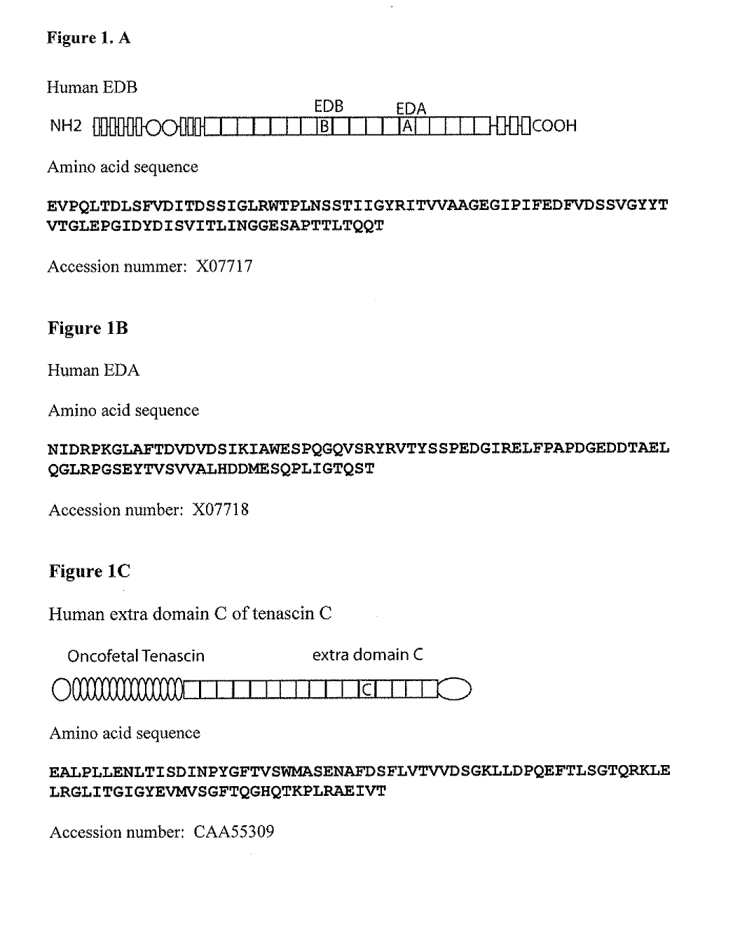

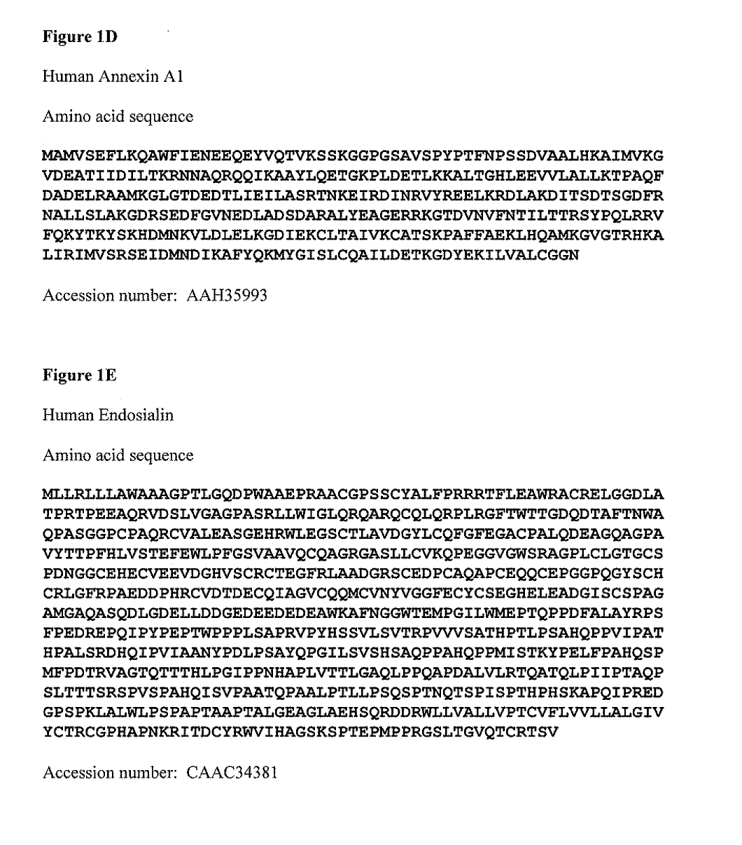

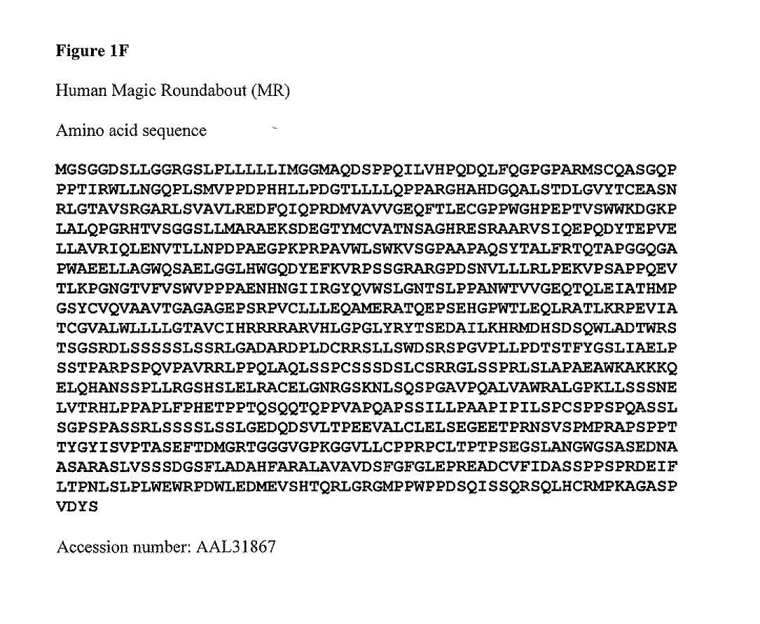

There are at least three different strategies to do these modifications of the self-protein. One method is to produce a fusion protein between a non-self protein, and the entire or a selected fragment of more than 5 amino acids of self EDB, tenacin C or MR in a prokaryotic or eukaryotic expression system. The open reading frame of EDB, EDA, annexin A1, endosialin, the extra domain C of tenascin C or magic roundabout (MR), as exemplified by human EDB, EDA, annexin A1, endosialin, the extra domain C of tenascin C or magic roundabout (MR) in FIG. 1, is then first cloned into a bacterial, fungal or eukaryotic vector. This fusion protein construct is then transfected into a mammalian or prokaryotic host for the production of the desired fusion protein. The fusion partner can here be any non-self protein of any size from 10 amino acids to several hundred kD. However, it is usually favorable to use a fusion partner of approximately the same size as the self-protein.

Alternatively, a non-modified EDB, EDA, annexin A1, endosialin, the extra domain C of tenascin C or magic roundabout (MR) can be produced in a mammalian or prokaryotic host or host cell line and then covalently attached to a carrier protein by chemical coupling.

A third alternative, which in our mind is less attractive, is to produce selected regions of the EDB, EDA, annexin A1, endosialin, the extra domain C of tenascin C or magic roundabout (MR) sequences as synthetic peptides and then to attach these peptides to a foreign carrier molecule by chemical coupling. This third alternative usually results, after injection into the patient, in antibody responses that show low binding activity against the native properly folded protein and thereby in lower clinical effect.

Following production the vaccine antigen is then purified and tested for pyrogen content and potential content of other contaminants. In order to obtain sufficiently strong immune response against the self-epitopes the vaccine antigen is then (optionally) mixed with an adjuvant before injection into the patient. After administration in the patient the vaccine induces an immune response against the vaccine antigen. Due to the presence of self-epitopes in the vaccine antigen this protein also induces an antibody response against the target molecule, here EDB, EDA, annexin A1, endosialin, the extra domain C of tenascin C or magic roundabout (MR), thereby targeting the immune system to attack the tumor vessels, which leads to reducing the growth of the tumor and maybe even total removal of the tumor.

### EXAMPLE

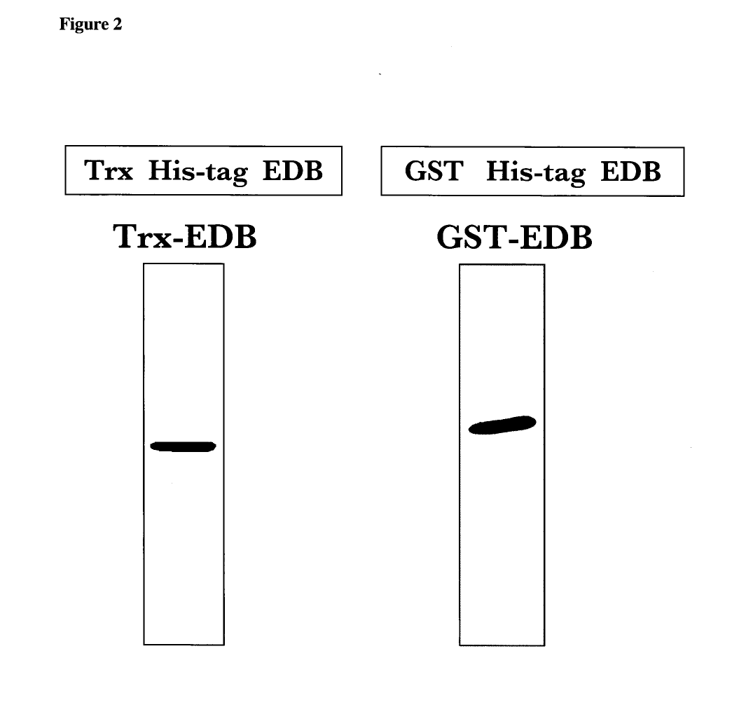

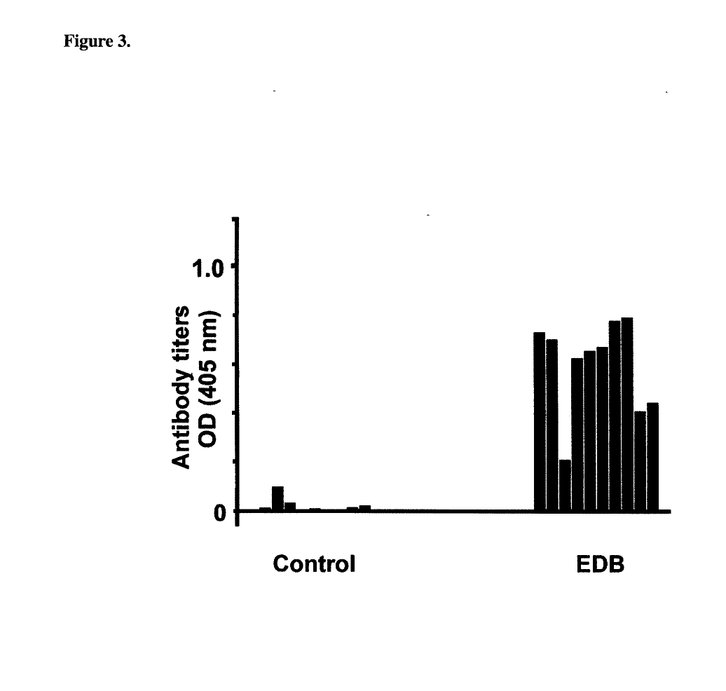

To test the efficacy of the invention a fusion protein between the 91 amino acid long extra-cellular domain B (EDB) of human and mouse fibronectin and a bacterial antigen of a size of approximately 10 kD, the E. coli thioredoxin was used as vaccine antigen to study the effect in an animal model. The EDB domain is very highly conserved and identical in almost in all placental mammals studied. This fusion protein was produced in a prokaryotic host to almost homogeneity (FIG. 2). The thioredoxin-EDB-fusion protein was then injected in mice together with an adjuvant. After three weeks the mice received a booster dose of the vaccine and after five weeks of treatment serum from these animals were tested for the amount of anti-EDB antibodies produced. As can be seen from FIG. 3 all animals showed high titers of anti-EDB antibodies whereas all the controls were negative. This shows that the vaccine has the capacity to induce production of substantial amounts of anti-EDB antibodies in a test animal.

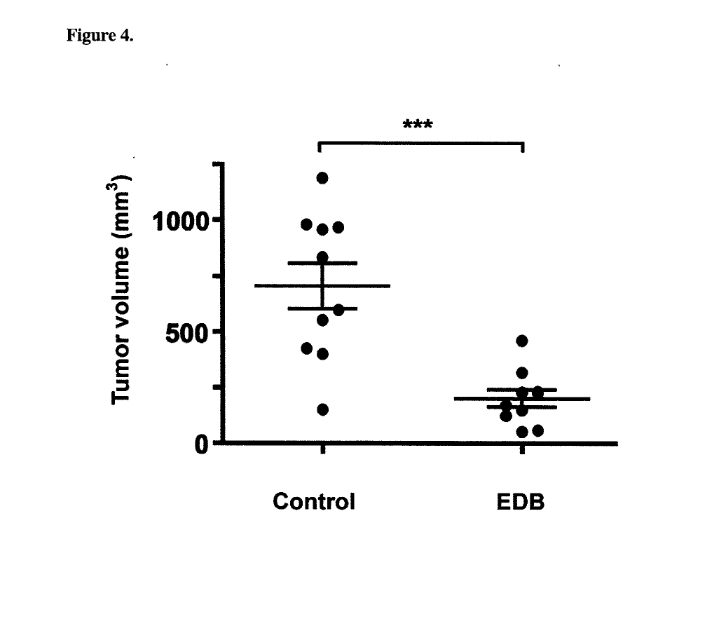

The in vivo efficacy of these antibodies was then tested in a mouse tumor-model. The vaccination and the antibodies produced after triggering the immune system of the host antibodies were found to effectively reduce the tumor size in these animals (FIG. 4). The anti EDB antibodies also resulted in a marked change in tissue of the tumor as observed by electron microscopic examination (data not shown). The binding of the anti-EDB antibodies to the tumor vasculature does clearly cause a marked infiltration of immune cells and an attack by the immune system on these vessels. It is most likely this effect on the vasculature that causes the potent reduction in tumor size observed in the vaccinated animals.

In conclusion, we show, that in contrast to most previous studies on cancer vaccines, both high titers and biological active antibodies induced by the vaccine and that it has good therapeutic effect on the tumor growth.

### REFERENCES

[1] Olsson A K, Dimberg A, Kreuger J, Claesson-Welsh L. VEGF receptor signalling—in control of vascular function. Nat Rev Mol Cell Biol 2006;7(5):359-71.

[2] Folkman J. Endogenous angiogenesis inhibitors. Apmis 2004;112(7-8):496-507.

[3] Hawighorst T, Velasco P, Streit M, Hong Y K, Kyriakides T R, Brown L F, et al. Thrombospondin-2 plays a protective role in multistep carcinogenesis: a novel host anti-tumor defense mechanism. Embo J 2001;20(11):2631-40.

[4] Sund M, Hamano Y, Sugimoto H, Sudhakar A, Soubasakos M, Yerramalla U, et al. Function of endogenous inhibitors of angiogenesis as endothelium-specific tumor suppressors. Proc Natl Acad Sci U S A 2005;102(8):2934-9.

[5] Ferrara N, Kerbel R S. Angiogenesis as a therapeutic target. Nature 2005 ;438(7070): 967-74.

[6] Scarpino S, Stoppacciaro A, Pellegrini C, Marzullo A, Zardi L, Tartaglia F, et al. Expression of EDA/EDB isoforms of fibronectin in papillary carcinoma of the thyroid. J Pathol 1999;188(2):163-7.

[7] Silacci M, Brack S S, Spath N, Buck A, Hillinger S, Arni S, et al. Human monoclonal antibodies to domain C of tenascin-C selectively target solid tumors in vivo. Protein Eng Des Sel 2006;19(10):471-8.

[8] Oh P, Li Y, Yu J, Durr E, Krasinska K M, Carver L A, et al. Subtractive proteomic mapping of the endothelial surface in lung and solid tumours for tissue-specific therapy. Nature 2004;429(6992):629-35.

[9] Teicher B A. Newer vascular targets: endosialin (review). Int J Oncol 2007;30(2):305-12.

[10] Rettig W J, Garin-Chesa P, Healey J H, Su S L, Jaffe E A, Old L I. Identification of endosialin, a cell surface glycoprotein of vascular endothelial cells in human cancer. Proc Natl Acad Sci U S A 1992;89(22):10832-6.

[11] Huminiecki L, Gorn M, Suchting S, Poulsom R, Bicknell R. Magic roundabout is a new member of the roundabout receptor family that is endothelial specific and expressed at sites of active angiogenesis. Genomics 2002;79(4):547-52.

1-25. (canceled)

26. Vaccine consisting of a single or a combination of the amino acid sequences of the extra domain B of fibronectin (EDB), the extra domain A of fibronectin (EDA), annexin A1, endosialin, the extra domain C of tenascin C, or magic roundabout (MR) or at least one fragment thereof, in its original or multimerized form, coupled to a carrier molecule, and a pharmaceutically acceptable adjuvant.

27. Vaccine according to claim 26, wherein the EDB, EDA, annexin A1, endosialin, the extra domain C of tenascin C, and magic roundabout (MR) is of human, canine, feline or equine origin.

28. An EDB, EDA, annexin A1, endosialin, extra domain C of tenascin C, and magic roundabout (MR) vaccine according to claim 26 for use in medicine.

29. An EDB, EDA, annexin A1, endosialin, extra domain C of tenascin C, and magic roundabout (MR) vaccine according to claim 27 for use in medicine.

30. The use of a fusion protein consisting of the entire or parts of EDB, EDA, annexin A1, endosialin, the extra domain C of tenascin C, or magic roundabout (MR) from the species to be treated and a foreign carrier protein for production of a vaccine for medical use.


SEQUENCE LISTING (6 sequences)

SEQ ID NO: 1 - protein, 91 aa, Homo sapiens
EVPQLTDLSFVDITDSSIGLRWTPLNSSTIIGYRITVVAAGEGIPIFEDFVDSSVGYYTVTGLEPGIDYDISVITLINGGESAPTTLTQQT

SEQ ID NO: 2 - protein, 90 aa, Homo sapiens
NIDRPKGLAFTDVDVDSIKIAWESPQGQVSRYRVTYSSPEDGIRELFPAPDGEDDTAELQGLRPGSEYTVSVVALHDDMESQPLIGTQST

SEQ ID NO: 3 - protein, 91 aa, Homo sapiens
EALPLLENLTISDINPYGFTVSWMASENAFDSFLVTVVDSGKLLDPQEFTLSGTQRKLELRGLITGIGYEVMVSGFTQGHQTKPLRAEIVT

SEQ ID NO: 4 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 5 - protein, 757 aa, Homo sapiens
MLLRLLLAWAAAGPTLGQDPWAAEPRAACGPSSCYALFPRRRTFLEAWRACRELGGDLATPRTPEEAQRVDSLVGAGPASRLLWIGLQRQARQCQLQRPLRGFTWTTGDQDTAFTNWAQPASGGPCPAQRCVALEASGEHRWLEGSCTLAVDGYLCQFGFEGACPALQDEAGQAGPAVYTTPFHLVSTEFEWLPFGSVAAVQCQAGRGASLLCVKQPEGGVGWSRAGPLCLGTGCSPDNGGCEHECVEEVDGHVSCRCTEGFRLAADGRSCEDPCAQAPCEQQCEPGGPQGYSCHCRLGFRPAEDDPHRCVDTDECQIAGVCQQMCVNYVGGFECYCSEGHELEADGISCSPAGAMGAQASQDLGDELLDDGEDEEDEDEAWKAFNGGWTEMPGILWMEPTQPPDFALAYRPSFPEDREPQIPYPEPTWPPPLSAPRVPYHSSVLSVTRPVVVSATHPTLPSAHQPPVIPATHPALSRDHQIPVIAANYPDLPSAYQPGILSVSHSAQPPAHQPPMISTKYPELFPAHQSPMFPDTRVAGTQTTTHLPGIPPNHAPLVTTLGAQLPPQAPDALVLRTQATQLPIIPTAQPSLTTTSRSPVSPAHQISVPAATQPAALPTLLPSQSPTNQTSPISPTHPHSKAPQIPREDGPSPKLALWLPSPAPTAAPTALGEAGLAEHSQRDDRWLLVALLVPTCVFLVVLLALGIVYCTRCGPHAPNKRITDCYRWVIHAGSKSPTEPMPPRGSLTGVQTCRTSV

SEQ ID NO: 6 - protein, 1007 aa, Homo sapiens
MGSGGDSLLGGRGSLPLLLLLIMGGMAQDSPPQILVHPQDQLFQGPGPARMSCQASGQPPPTIRWLLNGQPLSMVPPDPHHLLPDGTLLLLQPPARGHAHDGQALSTDLGVYTCEASNRLGTAVSRGARLSVAVLREDFQIQPRDMVAVVGEQFTLECGPPWGHPEPTVSWWKDGKPLALQPGRHTVSGGSLLMARAEKSDEGTYMCVATNSAGHRESRAARVSIQEPQDYTEPVELLAVRIQLENVTLLNPDPAEGPKPRPAVWLSWKVSGPAAPAQSYTALFRTQTAPGGQGAPWAEELLAGWQSAELGGLHWGQDYEFKVRPSSGRARGPDSNVLLLRLPEKVPSAPPQEVTLKPGNGTVFVSWVPPPAENHNGIIRGYQVWSLGNTSLPPANWTVVGEQTQLEIATHMPGSYCVQVAAVTGAGAGEPSRPVCLLLEQAMERATQEPSEHGPWTLEQLRATLKRPEVIATCGVALWLLLLGTAVCIHRRRRARVHLGPGLYRYTSEDAILKHRMDHSDSQWLADTWRSTSGSRDLSSSSSLSSRLGADARDPLDCRRSLLSWDSRSPGVPLLPDTSTFYGSLIAELPSSTPARPSPQVPAVRRLPPQLAQLSSPCSSSDSLCSRRGLSSPRLSLAPAEAWKAKKKQELQHANSSPLLRGSHSLELRACELGNRGSKNLSQSPGAVPQALVAWRALGPKLLSSSNELVTRHLPPAPLFPHETPPTQSQQTQPPVAPQAPSSILLPAAPIPILSPCSPPSPQASSLSGPSPASSRLSSSSLSSLGEDQDSVLTPEEVALCLELSEGEETPRNSVSPMPRAPSPPTTYGYISVPTASEFTDMGRTGGGVGPKGGVLLCPPRPCLTPTPSEGSLANGWGSASEDNAASARASLVSSSDGSFLADAHFARALAVAVDSFGFGLEPREADCVFIDASSPPSPRDEIFLTPNLSLPLWEWRPDWLEDMEVSHTQRLGRGMPPWPPDSQISSQRSQLHCRMPKAGASPVDYS